(19) United States
(12) Patent Application Publication
(10) Pub. No.: US 2026/0002937 A1
(43) Pub. Date: Jan. 1, 2026

(54) RAPID TEST STRIP

(71) Applicant: Quanta Vision Co., Ltd., New Taipei City (TW)

(72) Inventors: CHIH-YI HSU, New Taipei City (TW); YI-SHAN WENG, New Taipei City (TW); YA-CHIEH YU, New Taipei City (TW)

(21) Appl. No.: 19/252,587

(22) Filed: Jun. 27, 2025

(30) Foreign Application Priority Data
Jun. 28, 2024 (TW) 113124256

(51) Int. Cl. G01N 33/543; G01N 21/77; G01N 21/78; G01N 33/553

(52) U.S. Cl. CPC G01N 33/54388; G01N 21/78; G01N 33/553; G01N 2021/7759

(57) ABSTRACT

A rapid test strip is provided, wherein the rapid test strip includes a base layer, a sample pad, a first conjugate pad, a second conjugate pad, a reaction pad and an absorption pad, the sample pad, the first conjugate pad, the second conjugate pad, the reaction pad and the absorption pad are sequentially disposed on the base layer along a first direction, the first conjugate pad includes a plurality of gold-conjugated antibody complexes, and the second conjugate pad includes a plurality of gold-conjugated trithiocyanuric acid complexes.

### BACKGROUND OF THE INVENTION

### Field of the Invention

The present invention relates to a test strip, particularly to a rapid test strip.

### Description of the Prior Art

A lateral flow immunoassay rapid test reagent is a technique used for rapidly detecting specific biomolecules in a biological sample. It enables the detection of a small amount of biomolecules in a short time. The lateral flow immunoassay rapid test reagent is commonly used in fields such as clinical diagnostics, disease monitoring, biological research, and food safety. In a competitive lateral flow immunoassay rapid test reagent, the affinity between the antibody and antigen is a key factor in the development process. If the affinity is too weak or too strong, it may affect the test performance. When the affinity between the antibody and antigen does not meet expectations, the distinction between positive and negative results may not be apparent, thus affecting the accuracy of the result.

A conventional competitive lateral flow immunoassay rapid test reagent generally includes a sample pad, a conjugate pad, a rapid test membrane, and an absorption pad. A specimen first flows from the sample pad toward the absorption pad through the capillary action of fibers. When the specimen contains a target analyte, according to the principles of competitive or sandwich immunoassay, the test line will show either disappearance or appearance. However, when the antibody and antigen affinity is not as expected in the conventional competitive lateral flow immunoassay rapid test reagent, it may lead to difficulty in clearly distinguishing positive from negative results.

The present invention is, therefore, arisen to obviate or at least mitigate the above-mentioned disadvantages.

### SUMMARY OF THE INVENTION

The main object of the present invention is to provide a rapid test strip that offers a high-intensity detection signal.

To achieve the above and other objects, a rapid test strip is provided, wherein the rapid test strip includes a base layer, a sample pad, a first conjugate pad, a second conjugate pad, a reaction pad and an absorption pad, the sample pad, the first conjugate pad, the second conjugate pad, the reaction pad and the absorption pad are sequentially disposed on the base layer along a first direction, the first conjugate pad includes a plurality of gold-conjugated antibody complexes, and the second conjugate pad includes a plurality of gold-conjugated trithiocyanuric acid complexes.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a schematic structural diagram of an exemplary embodiment of the present invention;

FIG. 2 is a top view schematic structural diagram of an exemplary embodiment of the present invention;

FIG. 3 is a partially enlarged view of a first coating layer in FIG. 1;

FIG. 4 is a partially enlarged view of a second coating layer in FIG. 1;

FIG. 5 is an experimental result diagram showing detection of a specimen (BE) by the present invention and a graph showing the relationship between the specimen concentration and the difference value; and

FIG. 6 is an experimental result diagram of a conventional technology detecting a specimen (BE) and a graph showing the relationship between the specimen concentration and the difference value.

### DETAILED DESCRIPTION OF THE PREFERRED EMBODIMENTS

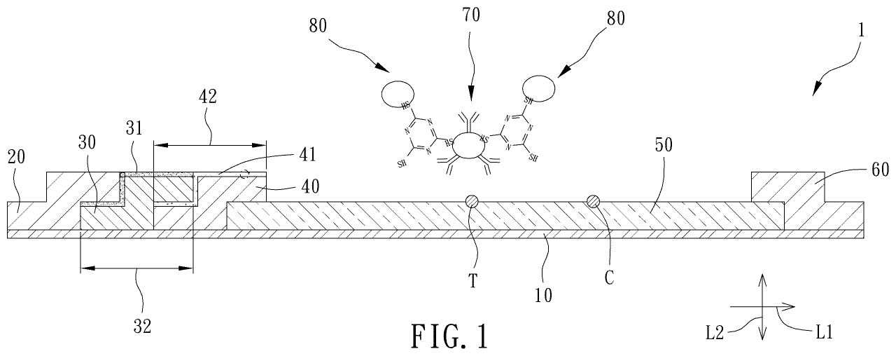

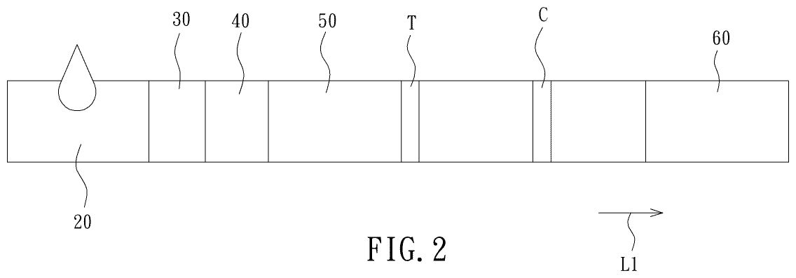

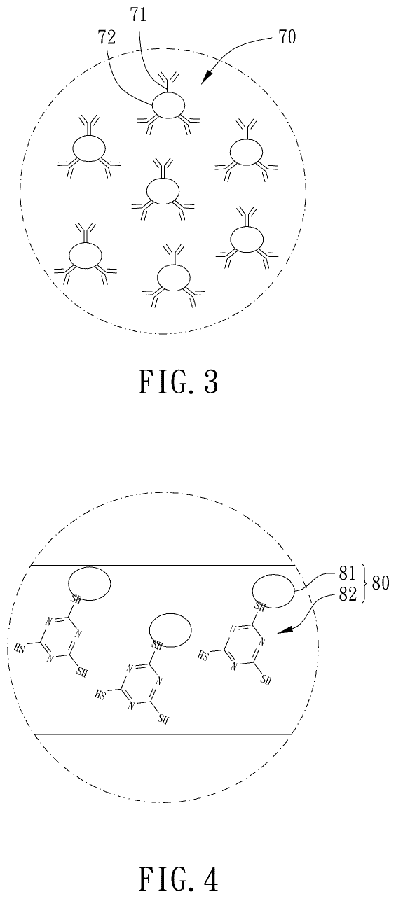

Please refer to FIGS. 1 to 5 for an exemplary embodiment of the present invention. A rapid test strip 1 of the present invention includes a base layer 10, a sample pad 20, a first conjugate pad 30, a second conjugate pad 40, a reaction pad 50, and an absorption pad 60.

The sample pad 20, the first conjugate pad 30, the second conjugate pad 40, the reaction pad 50 and the absorption pad 60 are sequentially disposed on the base layer 10 along a first direction L1. The first conjugate pad 30 includes a plurality of gold-conjugated antibody complexes 70, and the second conjugate pad 40 includes a plurality of gold-conjugated trithiocyanuric acid complexes 80. As such, a high-intensity detection signal is provided.

The base layer 10 may be a polyvinyl chloride (PVC) sheet or another type of plate.

The plurality of gold-conjugated antibody complexes 70 are disposed by spraying to form a first coating layer 31 covering the first conjugate pad 30. The plurality of gold-conjugated trithiocyanuric acid complexes 80 are disposed by spraying to form a second coating layer 41 covering the second conjugate pad 40. Preferably, an amount of a sprayed liquid of the plurality of gold-conjugated antibody complexes 70 on the first conjugate pad 30 is equal to an amount of a sprayed liquid of the plurality of gold-conjugated trithiocyanuric acid complexes 80 on the second conjugate pad 40. Specifically, the amount of the sprayed liquid of the plurality of gold-conjugated antibody complexes 70 on the first conjugate pad 30 and the amount of the sprayed liquid of the plurality of gold-conjugated trithiocyanuric acid complexes 80 on the second conjugate pad 40 are both 5.00±1.00 μL.

The second conjugate pad 40 is made of glass fiber material. The first conjugate pad 30 is made of glass fiber material.

In this embodiment, a first projection size 32 of the first conjugate pad 30 in a second direction L2 is equal to a second projection size 42 of the second conjugate pad 40 in the second direction L2, and the second direction L2 is perpendicular to the first direction L1.

The second conjugate pad 40 is stacked with the first conjugate pad 30 and the reaction pad 50, allowing the specimen to sequentially flow smoothly from the sample pad 20 through the first conjugate pad 30, the second conjugate pad 40, the reaction pad 50 and the absorption pad 60.

Each of the plurality of gold-conjugated trithiocyanuric acid complexes 80 is formed by bonding trithiocyanuric acid 82 with a conjugate 81 via sulfur-gold bonds. Each of the plurality of gold-conjugated antibody complexes 70 is bonded to the gold-conjugated trithiocyanuric acid complexes 80 via sulfur-gold bonds. Each of the plurality of gold-conjugated antibody complexes 70 is formed by bonding a conjugate 72 with a plurality of antibodies 71.

The reaction pad 50 is a nitrocellulose membrane, and the reaction pad 50 is sequentially disposed with a test line T and a control line C in a direction from the second conjugate pad 40 to the absorption pad 60.

During use, a specimen is dropped onto the sample pad 20. The specimen flows from the sample pad 20 toward the absorption pad 60 via capillary action. As the specimen flows through the first conjugate pad 30 and then the second conjugate pad 40, the plurality of gold-conjugated antibody complexes 70 bond with the plurality of gold-conjugated trithiocyanuric acid complexes 80. Each of the plurality of gold-conjugated antibody complexes 70 is bonded via sulfur-gold bonds to the gold-conjugated trithiocyanuric acid complexes 80.

As such, using the same amount of antibodies and antigens, the test line T exhibits two to three times the color development of the conjugate, thereby enhancing the difference between positive and negative detection results and reducing false positives and false negatives.

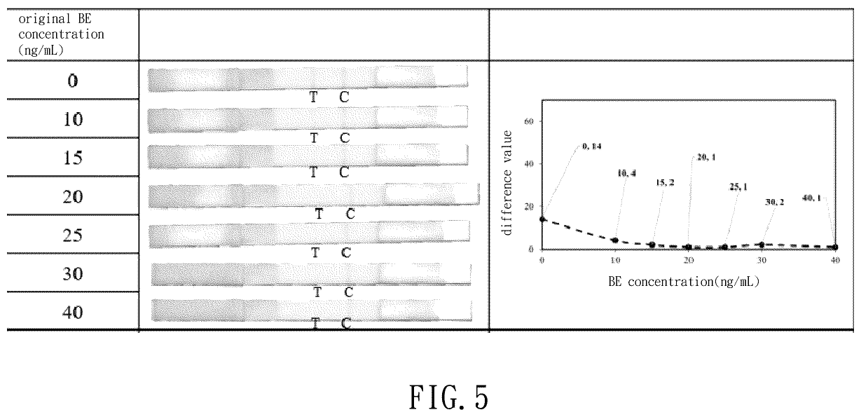

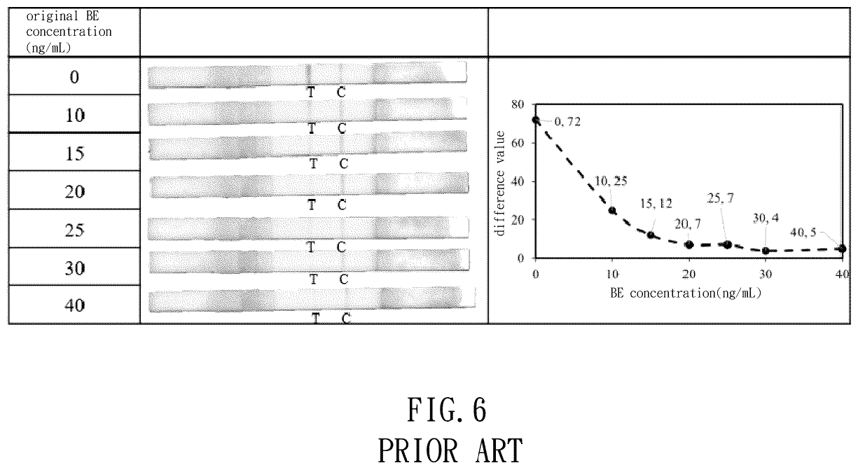

As shown in FIG. 6, test results of a conventional rapid test strip are presented. FIG. 5 shows test results of the rapid test strip 1 of the present invention. The experiments were conducted using a competitive immunoassay. Specifically, in this case (FIG. 5), as the concentration of the specimen (BE) increases (from 0 ng/ml to 40 ng/ml), the color of the test line T on the rapid test strip gradually fades. In FIG. 6 (conventional one), under a concentration of 0 ng/ml, the color of the test line T is already faint, and under concentrations below the threshold, the test line disappears, which can be misjudged as a positive result, thus leading to false positives. In contrast, in FIG. 5 representing the present invention, the difference between positive and negative results is apparent, making misjudgment less likely. Specifically, the detection threshold of the target analyte is 20 ng/ml (i.e., the legal detection concentration; if the concentration is above the threshold, it is determined to be positive; if below, it is determined to be negative). Moreover, according to the graph in FIG. 5 showing the relationship between the specimen (BE) concentration and the difference value in the present invention, compared to FIG. 6, the difference value of a similar concentration in the present invention is higher than that in the prior art. Therefore, the rapid test strip 1 of the present invention offers a high-intensity detection signal.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A rapid test strip including a base layer, a sample pad, a first conjugate pad, a second conjugate pad, a reaction pad and an absorption pad, wherein the sample pad, the first conjugate pad, the second conjugate pad, the reaction pad and the absorption pad are sequentially disposed on the base layer along a first direction, the first conjugate pad includes a plurality of gold-conjugated antibody complexes, and the second conjugate pad includes a plurality of gold-conjugated trithiocyanuric acid complexes.

2. The rapid test strip of claim 1, wherein the plurality of gold-conjugated antibody complexes are disposed by spraying to form a first coating layer covering the first conjugate pad.

3. The rapid test strip of claim 2, wherein the plurality of gold-conjugated trithiocyanuric acid complexes are formed by spraying and disposed over a second coating layer of the second conjugate pad.

4. The rapid test strip of claim 1, wherein the second conjugate pad is made of glass fiber material.

5. The rapid test strip of claim 1, wherein an amount of a sprayed liquid of the plurality of gold-conjugated antibody complexes on the first conjugate pad is equal to an amount of a sprayed liquid of the plurality of gold-conjugated trithiocyanuric acid complexes on the second conjugate pad.

6. The rapid test strip of claim 1, wherein the second conjugate pad is stacked with the first conjugate pad and the reaction pad.

7. The rapid test strip of claim 1, wherein a first projection size of the first conjugate pad in a second direction is equal to a second projection size of the second conjugate pad in the second direction, and the second direction is perpendicular to the first direction.

8. The rapid test strip of claim 1, wherein each of the plurality of gold-conjugated trithiocyanuric acid complexes is formed by bonding trithiocyanuric acid with a conjugate via sulfur-gold bonds.

9. The rapid test strip of claim 1, wherein each of the plurality of gold-conjugated antibody complexes is bonded to the gold-conjugated trithiocyanuric acid complexes via sulfur-gold bonds.

10. The rapid test strip of claim 3, wherein: the second conjugate pad is made of glass fiber material; the first conjugate pad is made of glass fiber material; an amount of a sprayed liquid of the plurality of gold-conjugated antibody complexes on the first conjugate pad is equal to an amount of a sprayed liquid of the plurality of gold-conjugated trithiocyanuric acid complexes on the second conjugate pad; the second conjugate pad is stacked with the first conjugate pad and the reaction pad; a first projection size of the first conjugate pad in a second direction is equal to a second projection size of the second conjugate pad in the second direction, and the second direction is perpendicular to the first direction; each of the plurality of gold-conjugated trithiocyanuric acid complexes is formed by bonding of a sulfur gold bond and conjugate bond; the plurality of gold-conjugated antibody complexes are bonded to the plurality of gold-conjugated trithiocyanuric acid complexes via sulfur-gold bonds;
the reaction pad is a nitrocellulose membrane, and the reaction pad is sequentially disposed with a test line and a control line in a direction from the second conjugate pad to the absorption pad; the amount of the sprayed liquid of the plurality of gold-conjugated antibody complexes on the first conjugate pad and the amount of the sprayed liquid of the plurality of gold-conjugated trithiocyanuric acid complexes on the second conjugate pad are both 5.00±1.00 μL.
